(19) United States
(12) Patent Application Publication
(10) Pub. No.: US 2024/0382408 A1
(43) Pub. Date: Nov. 21, 2024

(54) COSMETIC COMPOSITION FOR USE ON THE SKIN

(71) Applicant: Timm Golüke, München (DE)

(72) Inventor: Timm Golüke, München (DE)

(21) Appl. No.: 18/692,547

(22) PCT Filed: Sep. 8, 2022
(86) PCT No.: PCT/EP2022/074952

§ 371 Date: Mar. 15, 2024

(30) Foreign Application Priority Data
Sep. 17, 2021 (DE) 10 2021 124 078.6

(51) Int. Cl. A61K 8/9749; A61K 8/14; A61K 8/34; A61K 8/55; A61Q 19/00

(52) U.S. Cl. CPC A61K 8/9749; A61K 8/14; A61K 8/345; A61K 8/553; A61Q 19/00

(57) ABSTRACT

A cosmetic composition for application to the skin comprising a first ingredient (1) which comprises the aerial plant parts of the royal fern, water and polyhydric alcohols, and further comprising a second ingredient (2) which comprises a liposomally encapsulated form of the first ingredient (1).

The invention pertains to a cosmetic composition for application to the skin.

The invention pertains to the technical field of industrially manufactured cosmetic compositions, in particular those cosmetic compositions for the manufacture of which natural substances in the form of plant extracts are used.

The cosmetics industry is constantly launching new products on the market, which in many cases contain a large number of synthetically produced raw materials that cause skin irritation in users with sensitive skin, sometimes already during the first application attempts, but often also during long-term use. In recent years, natural substances in the form of plant extracts have been increasingly offered and used in cosmetics. However, in most cases these products only have a very specific effect, which is not always sufficient.

The cosmetics industry is increasingly focusing on natural substances and bioactive ingredients that are obtained using biotechnological processes. They not only fulfil the consumer's desire for bio-based, more sustainable products, but also exhibit completely new properties and effect mechanisms.

Plant-based raw materials have been an important source of cosmetic products since the early days of humanity. This is still the case today, except that the possibilities of biotechnology have made it possible to utilize ingredients that could not be extracted or produced in the necessary quantities just a few years ago. At the same time, the industry is endeavoring to obtain ingredients from biological resources in the future that were previously obtained using petrochemical processes. This promises greater sustainability and new product properties.

EP2752193, for example, discloses a cosmetic that contains a natural substance complex as well as other substances for skin care.

One active ingredient complex that fulfils all the important tasks of skin care is the royal fern complex, which is beneficial for strengthening and supporting the skin's resistance. The flowering fern (osmunda regalis), better known by its major name “royal fern”, is the only species of the royal fern genus (osmunda) native to Europe. Like other royal fern species, it belongs to the royal fern family (osmundaceae). It prefers to grow in mild winter regions and is a protected species in Germany due to the severe decline seen in its natural habitats. Therefore, it is prohibited to excavate the roots.

The royal fern is known as a medicinal plant for internal and external use. The active parts are both the leaves and the roots. The pharmaceutical industry uses ingredients from this plant. The tannins contained in the rootstock of the royal fern, when applied as decoction-soaked compresses, promote wound healing for minor injuries, abrasions and light burns. The following effects are also attributed to the royal fern: a laxative, an astringent and a diuretic effect.

A drawback of the cosmetic compositions of the prior art is that they contain all the active ingredients of the royal fern, i.e. also the roots, in order to achieve sufficient moisturization of the skin.

Thus, the object of the invention is to provide a cosmetic composition for application to the skin which overcomes the drawbacks of the prior art and brings about improved moisturization of the skin without the use of protected plant parts.

This object is achieved by a cosmetic composition according to claim 1. Advantageous aspects constitute the subject-matter of the respective subclaims.

The invention encompasses a cosmetic composition for application to the skin. The cosmetic composition comprises a first ingredient comprising the aerial parts of the royal fern, water and polyhydric alcohols. Further, the cosmetic composition comprises a second ingredient comprising a liposomally encapsulated form of the first ingredient. This enables the production of highly effective natural cosmetics using the leaves and stalks of the royal fern, so that the protected rootstocks can remain in the soil and the moisturization of the skin is still unexpectedly high.

An advantageous aspect provides that the second ingredient contains a content of 1 to 10% by weight, preferably 3 to 7% by weight, particularly preferably 5% by weight of lecithin. This makes it possible to produce an emulsion by combining the lipophilic and hydrophilic moieties of the cosmetic composition.

It is particularly advantageous if the lecithin is a sunflower lecithin. This enables domestic plants to be used to produce the lecithin and the use of soya, for example, can be dispensed with.

According to a preferred aspect, a proportion of 20% by weight of the first ingredient is present liposomally encapsulated in the lecithin of the second ingredient. This causes, inter alia, a reduction of hyperpigmentation of the skin.

According to an advantageous aspect, the polyhydric alcohols comprise glycerol and pentylene glycol. This results in a particularly high moisturizing effect on the skin and additionally stimulates collagen production in the skin.

It is particularly advantageous if the aerial plant parts of the royal fern include the stalk and the leaves of the plant. This enables the cosmetics to be produced sustainably while taking nature conservation into account.

Advantageously, the first ingredient is produced in a maceration process. This means that only the water-soluble, cosmetically active substances are dissolved, which strengthen the skin's resistance to harmful environmental influences such as pollution or UV radiation.

It has been found to be advantageous if the content of the first ingredient comprises a range from 0.01 to 1.0% by weight, preferably a range from 0.1 to 0.8% by weight, particularly preferably 0.5% by weight. This prevents the degradation of collagen and elastin in the skin.

An advantageous aspect provides that the content of the second ingredient comprises a range from 0.01 to 1.0% by weight, preferably a range from 0.1 to 0.8% by weight, particularly preferably 0.5% by weight. This enables particularly good handling of the ingredients in the manufacture of the cosmetic composition.

It is particularly advantageous if the particle size of the second ingredient is in a range from 50 to 300 nm, preferably in a range from 100 to 250 nm, particularly preferably 200 nm. This results in an optimum surface-to-volume ratio so that the active ingredients can be dissolved out during further processing.

Advantageously, the lecithin content comprises a range from 0.01 to 0.5% by weight, preferably from 0.02 to 0.1% by weight, particularly preferably 0.025% by weight. This prevents the skin from drying out and thus represents a further positive effect of the cosmetic composition.

The invention further encompasses a process for preparing a first ingredient of such a cosmetic composition in a maceration process comprising the steps of
a. combining and homogenizing water, polyhydric alcohols and areal plant parts of the royal fern at 20° C. to 37° C., preferably at 24° C. to 30° C., particularly preferably at 25° C. for 1 to 3 minutes by comminuting the areal plant parts of the royal fern to a uniform particle size to form a precursor of the first ingredient
b. maceration of the first ingredient for at least 24 hours
c. filtering of the first ingredient via a pointed filter, whereby the mesh size is 0.05 to 0.3 mm, preferably 0.1 to 0.25 mm, particularly preferably 0.2 mm.

This makes it possible to dissolve the active substances from the areal plant parts of the royal fern, such as tannins.

Particularly advantageous is a process for the preparation of a second ingredient of such a cosmetic composition, in a process comprising the steps of
a. combining and mixing water, a polyhydric alcohol and xanthan gum under vacuum for 1 to 3 minutes
b. adding the first ingredient, a polyhydric alcohol and lecithin
c. homogenization under vacuum for at least one minute
d. cooling
e. homogenization under vacuum for 6 to 10 minutes at a maximum of 37° C.

This makes it possible to produce a homogeneous emulsion.

The invention is explained in more detail below with reference to one drawing.

In the drawing:

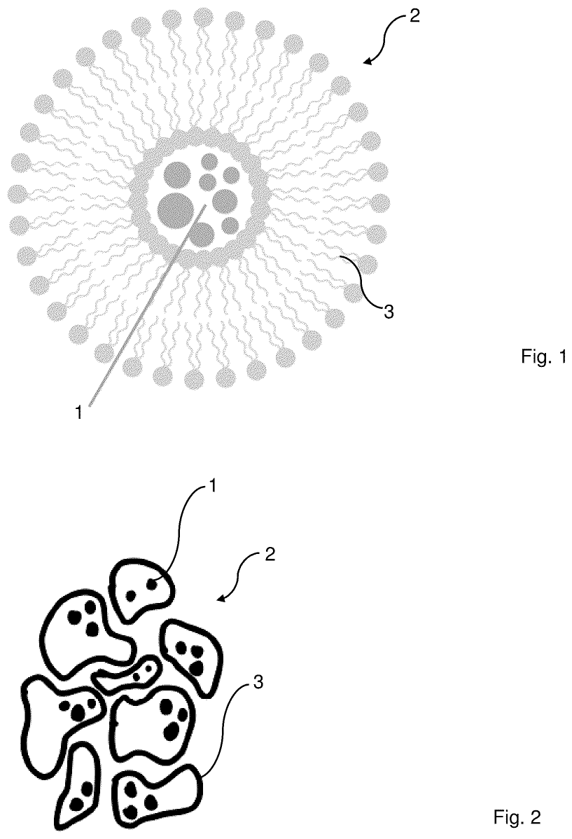

FIG. 1 shows a detailed view of a second ingredient of a cosmetic composition according to the invention with a liposomally encapsulated first ingredient; and

FIG. 2 shows a schematic view of a second ingredient of a cosmetic composition according to the invention with a liposomally encapsulated first ingredient.

FIG. 1 shows a detailed view of a second ingredient 2 of a cosmetic composition according to the invention with a liposomally encapsulated first ingredient 1. The cosmetic composition for application to the skin comprises a first ingredient 1, which comprises the aerial plant parts of the royal fern, water and polyhydric alcohols. Furthermore, the cosmetic composition comprises a second ingredient 2, which comprises a liposomally encapsulated form of the first ingredient 1. For example, the water content of the cosmetic composition is in a range from 60 to 80% by weight, preferably between 65 to 77% by weight, more preferably 75.5% by weight.

The second ingredient 2 contains a content of 1 to 10% by weight, preferably 3 to 7% by weight, particularly preferably 5% by weight of lecithin 3. The lecithin 3 is a sunflower lecithin. It is also conceivable to use other phospholipids as emulsifiers.

A proportion of 20% by weight of the first ingredient 1 is present liposomally encapsulated in the lecithin 3 of the second ingredient 2. The polyhydric alcohols include glycerol and pentylene glycol.

The areal plant parts of the royal fern comprise the stalk and the leaves of the plant.

FIG. 2 shows a schematic view of a second ingredient 2 of a cosmetic composition according to the invention with a liposomally encapsulated first ingredient 1. The first ingredient 1 is produced in a maceration process. The content of the first ingredient 1 comprises a range from 0.01 to 1.0% by weight, preferably a range from 0.1 to 0.8% by weight, particularly preferably 0.5% by weight.

The content of the second ingredient 2 comprises a range from 0.01 to 1.0% by weight, preferably a range from 0.1 to 0.8% by weight, particularly preferably 0.5% by weight.

The particle size of the second ingredient 2 is in a range from 50 to 300 nm, preferably in a range from 100 to 250 nm, particularly preferably 200 nm.

The content of lecithin 3 is in a range from 0.01 to 0.5% by weight, preferably from 0.02 to 0.1% by weight, particularly preferably 0.025% by weight.

A process for the preparation of a first ingredient 1 of a cosmetic composition as mentioned above in a maceration process comprises the steps of
a. combining and homogenizing water, polyhydric alcohols and areal plant parts of the royal fern at 20° C. to 37° C., preferably at 24° C. to 30° C., particularly preferably at 25° C. for 1 to 3 minutes by comminuting the areal plant parts of the royal fern to a uniform particle size to form a precursor of the first ingredient 1
b. maceration of the first ingredient 1 for at least 24 hours
c. filtering of the first ingredient 1 via a pointed filter, wherein the mesh size is 0.05 to 0.3 mm, preferably 0.1 to 0.25 mm, particularly preferably 0.2 mm.

A process for the preparation of a second ingredient 2 of the cosmetic composition as mentioned above comprises the steps of
a. combining and mixing water, a polyhydric alcohol and xanthan gum under vacuum for 1 to 3 minutes
b. adding the first ingredient 1, a polyhydric alcohol and lecithin 3
c. homogenization under vacuum for at least one minute
d. cooling
e. homogenization under vacuum for 6-10 minutes at a maximum of 37° C.

1. Cosmetic composition for application to the skin comprising a first ingredient comprising the aerial plant parts of the royal fern, water and polyhydric alcohols, and further comprising a second ingredient comprising a liposomally encapsulated form of the first ingredient.

2. Cosmetic composition according to claim 1, wherein the second ingredient contains a content of 1 to 10% by weight, preferably of 3 to 7% by weight, particularly preferably of 5% by weight of lecithin.

3. Cosmetic composition according to claim 1, wherein the lecithin is a sunflower lecithin.

4. Cosmetic composition according to claim 1, wherein a proportion of 20% by weight of the first ingredient is present liposomally encapsulated in the lecithin of the second ingredient.

5. Cosmetic composition according to claim 1, wherein the polyhydric alcohols comprise glycerol and pentylene glycol.

6. Cosmetic composition according to claim 1, wherein the aerial plant parts of the royal fern comprise the stalk and the leaves of the plant.

7. Cosmetic composition according to claim 1, wherein the first ingredient is prepared in a maceration process.

8. Cosmetic composition according to claim 1, wherein the content of the first ingredient comprises a range from 0.01 to 1.0% by weight, preferably a range from 0.1 to 0.8% by weight, more preferably 0.5% by weight.

9. Cosmetic composition according to claim 1, wherein the content of the second ingredient comprises a range from 0.01 to 1.0% by weight, preferably a range from 0.1 to 0.8% by weight, more preferably 0.5% by weight.

10. Cosmetic composition according to claim 1, wherein the particle size of the second ingredient is in a range from 50 to 300 nm, preferably in a range from 100 to 250 nm, more preferably 200 nm.

11. Cosmetic composition according to claim 1, wherein the content of lecithin comprises a range from 0.01 to 0.5% by weight, preferably from 0.02 to 0.1% by weight, more preferably 0.025% by weight.

12. Process for the preparation of a first ingredient of a cosmetic composition according to claim 1, in a maceration process comprising the steps of
a. combining and homogenizing water, polyhydric alcohols and aerial plant parts of the royal fern at 20° C. to 37° C., preferably at 24° C. to 30° C., particularly preferably at 25° C. for 1 to 3 minutes by comminuting the aerial plant parts of the royal fern to a uniform particle size to form a precursor of the first ingredient
b. maceration of the first ingredient for at least 24 hours
c. filtering of the first ingredient via a pointed filter, the mesh size being 0.05 to 0.3 mm, preferably 0.1 to 0.25 mm, particularly preferably 0.2 mm.

13. Process for the preparation of a second ingredient of a cosmetic composition according to claim 1, in a process comprising the steps of
a. combining and mixing water, a polyhydric alcohol and xanthan gum under vacuum for 1-3 minutes
b. adding the first ingredient, a polyhydric alcohol and lecithin
c. homogenization under vacuum for at least one minute
d. cooling
e. homogenization under vacuum for 6 to 10 minutes at a maximum of 37° C.
